(19) United States
(12) Patent Application Publication
(10) Pub. No.: US 2025/0099211 A1
(43) Pub. Date: Mar. 27, 2025

(54) Multispectral Intra-Oral Diagnostic Scanner

(71) Applicant: D4D Technologies, LLC, Richardson, TX (US)

(72) Inventors: Glen Freeman, Keller, TX (US); Ye Li, Plano, TX (US)

(21) Appl. No.: 18/471,915

(22) Filed: Sep. 21, 2023

(51) Int. Cl. A61C 9/00; A61B 1/00; A61B 1/06; A61B 1/24; G06T 15/20; G16H 30/40

(52) U.S. Cl. CPC A61C 9/006; A61B 1/000096; A61B 1/063; A61B 1/0638; A61B 1/0684; A61B 1/24; G06T 15/20; G16H 30/40; G06T 2210/41

(57) ABSTRACT

Systems and methods for performing multispectral intra-oral and other diagnostic scans of a physical object are disclosed. A handheld scanning device separately illuminates the physical object with a plurality of light sources and captures corresponding images of the physical object, which may comprise a patient's tooth, intra-oral tissue, or skin. The light sources, such as laser diodes and light-emitting diodes, use wavelengths selected for diagnostic and three-dimensional modeling efficacy, which may be based on translucence and fluorescence characteristics in objects being observed. The images, and a three-dimensional model derived therefrom potentially substantially immediately thereafter, form a diagnostic rendering that may be stored, presented, and analyzed to design and evaluate medical treatment plans. Additional images, such as x-ray images obtained from other instruments, may be added to the rendering, to help more completely inform patients and care providers of the patient's diagnostic situation. Patient care may thus be improved.

### TECHNICAL FIELD

This disclosure relates generally to optical scanning devices.

### BACKGROUND

Commonly-assigned U.S. Pat. No. 7,978,892B2 and 11,648,095B2 and U.S. Patent Application Publication 2023/0233295A1 describe handheld intra-oral scanning devices, and are incorporated into this disclosure by reference in their entirety. These references each provide an apparatus that projects structured patterns of light onto a nearby physical object, such as a patient's tooth or other dental item. Multiple cameras capture images that are then analyzed to build a three-dimensional model of the physical object via analysis of the structured patterns in the imagery and photogrammetry. The model may be used to design restorative dental devices. More recently, commonly-assigned U.S. patent application Ser. No. 18/455,027 filed on Aug. 24, 2023 and entitled “Holographic Digitizer Illuminator With Mutually Variant Wavelengths,” also incorporated into this disclosure by reference in its entirety, described an apparatus that instead uses holographic techniques to build a three-dimensional model of a physical object.

### BRIEF SUMMARY

In one embodiment, an apparatus for producing a diagnostic rendering of a patient's anatomy comprises an illuminator configured to generate electromagnetic radiation of a plurality of wavelengths selected for at least one of diagnostic and modeling characteristics, at least one imaging sensor configured to capture images of the patient's anatomy when illuminated with the selected wavelengths, and a modeling engine configured to generate a three-dimensional model of the patient's anatomy using at least some of the captured images and to assemble the diagnostic rendering from at least some of the captured images and the model.

In one embodiment, a method for producing a diagnostic rendering of a patient's anatomy comprises selecting a plurality of wavelengths of electromagnetic radiation for at least one of diagnostic and modeling characteristics, capturing images of the patient's anatomy when illuminated using the selected wavelengths, generating a three-dimensional model of the patient's anatomy using at least some of the captured images, and assembling the diagnostic rendering from at least some of the captured images and the model.

In one embodiment, a system for producing a diagnostic rendering of a patient's anatomy comprises means for selecting a plurality of wavelengths of electromagnetic radiation for at least one of diagnostic and modeling characteristics, means for capturing images of the patient's anatomy when illuminated using the selected wavelengths, means for generating a three-dimensional model of the patient's anatomy using at least some of the captured images, and means for assembling the diagnostic rendering from at least some of the captured images and the model.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document. For a more complete understanding of the disclosed subject matter and the advantages thereof, reference is now made to the following descriptions taken in conjunction with the accompanying drawings, in which:

FIG. 1 depicts a basic view of an embodiment of a conventional intra-oral scanner system 100;

FIG. 2 depicts an embodiment of a conventional illuminator 200 for an intra-oral scanner;

FIG. 3 depicts a side view of an embodiment of a multispectral scanner 300 according to this disclosure;

FIG. 4 depicts a top view of an embodiment of a multispectral scanner 300 according to this disclosure;

FIG. 5 depicts a flowchart 500 describing the operation of the scanning system in which the techniques of this disclosure may be implemented;

FIG. 6 depicts a computing component 600 that may carry out the functionality according to this disclosure.

### DETAILED DESCRIPTION OF THE EMBODIMENTS

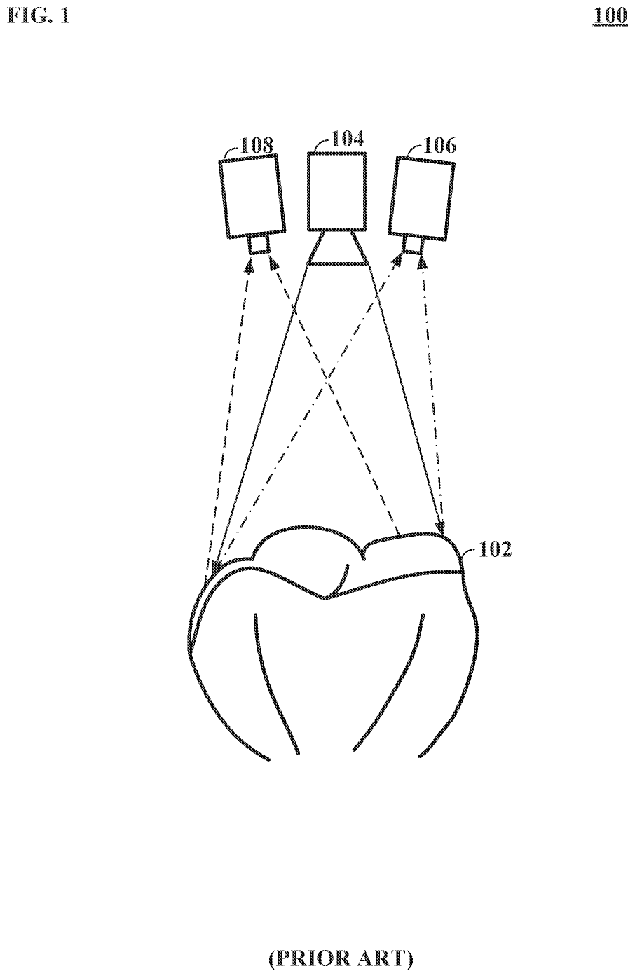

FIG. 1 depicts a basic view of an embodiment of a conventional intra-oral scanning system 100. A physical object 102, such as a tooth as shown, is illuminated by radiation source 104. A commercially-available DLP (digital light processing) micromirror array, not shown here for clarity, controls the production of the patterned light used in the production of three-dimensional models.

Imaging sensors 106 and 108 capture images of illuminated physical object 102 from slightly different angles. Imaging sensors 106 and 108 are preferably identical, so that the differences in images are due solely to geometry. Historically, film cameras were used as imaging sensors, but digital devices such as high resolution charge coupled devices (CCDs) and CMOS cameras are increasingly used today.

The data from the captured images is used to computationally develop a three-dimensional model of physical object 102. For example, photogrammetric triangulation techniques may be combined with the use of projected patterned light to produce a three-dimensional cloud of points on the surface of physical object 102. In dental applications, this three-dimensional model may be used to determine the precise shape of a patient's tooth or of dental appliances, such as crowns.

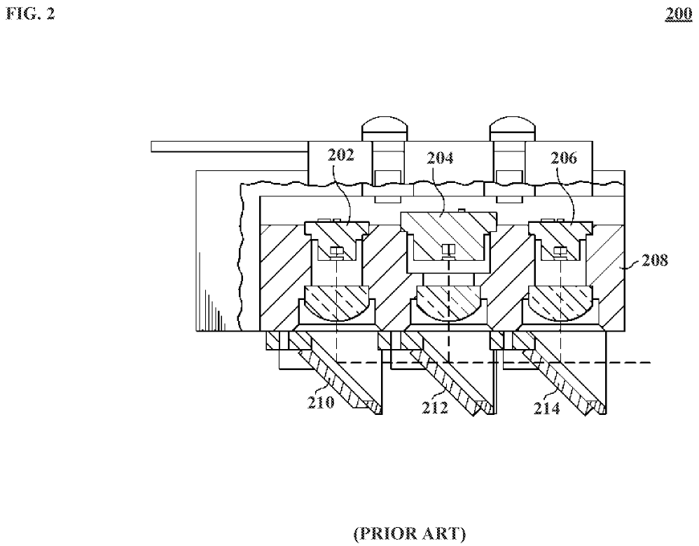

FIG. 2 depicts an embodiment of a conventional illuminator 200 for an intra-oral scanner. This illuminator or light engine module comprises three exemplary laser diodes, 202, 204, and 206, mounted in a housing 208. Housing 208 serves as a heat sink.

In one embodiment, the laser diodes emit red (202), green (204), and blue (206) light. A full-spectrum mirror 210, a red-passing and green-reflecting dichroic mirror 212, and a blue-reflecting red-and-green-passing dichroic mirror 214, respectively, are positioned near the laser diodes. The result is that the radiation emitted by the different laser diodes is transmitted along a common direction, to be patterned and directed toward physical object 102. The power output of each laser diode may be adjusted to achieve a balanced full-color (i.e., white light) image of physical object 102.

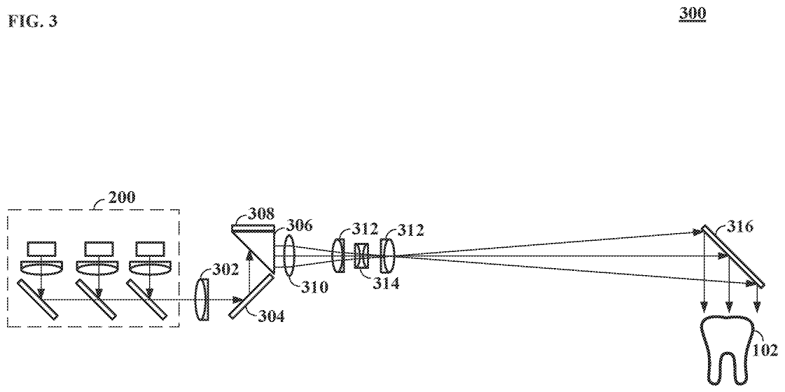

FIG. 3 depicts a side view of an embodiment of a multispectral scanner 300 according to this disclosure. One embodiment of a handheld scanning device described herein features a plurality of light sources, such as with illuminator 200, to illuminate a nearby physical object 102, such as a patient's tooth or other object of interest. Note that any number of light sources may be used in the illuminator, including any mixture of laser diodes and light-emitting diodes (LEDs). At least one imaging sensor (not shown in this figure, for clarity) may capture images of the physical object. The disclosed apparatus may also build a three-dimensional model of the physical object from the images.

All of the components of scanner 300 may be installed in a single housing for convenient use. Alternatively, some components such as the light sources may be installed in a separate housing that may be connected mechanically, electrically, and/or optically via optical fibers to a projection and imaging device often termed a “scanner head” or a “scanning tip”. See, for example, U.S. Pat. No. 11,648,095B2, U.S. Patent Application 2003/0233295A1, and U.S. patent application Ser. No. 18/455,027 previously mentioned.

Radiation from illuminator 200 proceeds through a first lens 302 to a first mirror 304, where it is reflected upward toward a total internal reflection prism assembly 306 in one embodiment. Assembly 306 transfers light that reflects from activated micromirrors in a DLP spatial modulator chip 308 outward to a projection path. Light from non-activated micromirrors in chip 308 is not transferred, thus chip 308 may produce a patterned light field to assist with generation of a three-dimensional model of physical object 102. Note that in contrast to the prior art, chip 308 is positioned on the top of prism assembly 306.

Transferred radiation proceeds through lens 310 into a set of projector lenses comprising two lenses 312 with an achromatic doublet lens 314 in between, in one embodiment. The projected radiation then travels to a second mirror 316, where it is directed downward toward physical object 102. In one embodiment, mirror 316 via which light is transmitted for illumination of physical object 102 and then received for imaging of physical object 102 is heated to prevent fogging.

Physical object 102 may comprise other portions of a patient's anatomy than a tooth. For example, other oral tissues, areas of skin or other soft tissues, and non-dental bones that may be visible during surgery may be advantageously observed and modeled. Physical object 102 may also comprise an article of manufacture being inspected.

In contrast to the prior art, scanner 300 of the present disclosure teaches advantageously using light of multiple spectral ranges, with each wavelength range chosen for particular diagnostic, investigative, and/or modeling purposes. For example, blue light at a wavelength of approximately 450 nanometers may be used to produce images of the surface of a tooth for the purpose of creating a three-dimensional model of the tooth. Such images are superior for surface modeling purposes because teeth are generally less translucent to blue light than to longer wavelengths, so there is less volume scattering. Violet light at a wavelength of approximately 405 nanometers, for example, may also be well-suited to this purpose for the same reason.

Conversely, light that is in the far red or near infrared wavelength range can penetrate more deeply into a tooth because of such translucence and volume scattering. Images made using such longer wavelengths, e.g., approximately 670 to 750 nanometers, are thus superior for providing indications for the diagnosis of defects that generally extend below the surface of a tooth, such as dental caries and cracks.

Such images can also be made of the skin or soft tissues of a patient, which are also relatively translucent to such longer-wavelength spectra. In one embodiment, such a scan may be used for the examination of scar tissue. Such images can show the progression of sub-surface tissue healing in burn victims and surgical patients over time.

Ultraviolet light that illuminates a physical object can cause fluorescence, a phenomenon in which light is responsively emitted at various longer wavelengths. The incoming ultraviolet stimulus light may be filtered out during imaging, such that substantially only the fluorescence-based light reaches an imaging sensor. Such fluorescence-based light may help diagnostically discern a healthy tooth, tartar or plaque or bacterial buildup, caries, and tooth demineralizations, for example.

The light sources in some embodiments may comprise LEDs or laser diodes that are each designed to produce spectra having a relatively narrow wavelength range, from infrared through ultraviolet. In other embodiments, LEDs that output a broad spectrum of substantially white light may be employed, to capture full-spectrum images or to be filtered into various desired narrower wavelength range spectra for separate imaging. LEDs generally do not emit coherent light, but have the advantages over laser diodes that they do not require despeckling apparatus, and may typically output more light per unit input power and per unit cost. In one embodiment, high power 1 mm×1 mm single die red, blue, and green LEDs may comprise the best mode for providing the maximum brightness. Single-piece LEDs that produce substantially white light are also available on the market.

Each wavelength range that may be emitted by each light source may be used to illuminate physical object 102 at separate times, to produce a number of images with only that illumination applied. In the case of capturing full-spectrum images, however, multiple light sources may be emitting light simultaneously. In the case of capturing fluorescence-based images, a stimulus wavelength may be filtered out of the images, leaving only the light due to the fluorescence in the images. The time required to separately illuminate physical object 102 with each wavelength and image it may be very brief, e.g., milliseconds per wavelength, so a full range of illuminations and corresponding images may be captured substantially immediately.

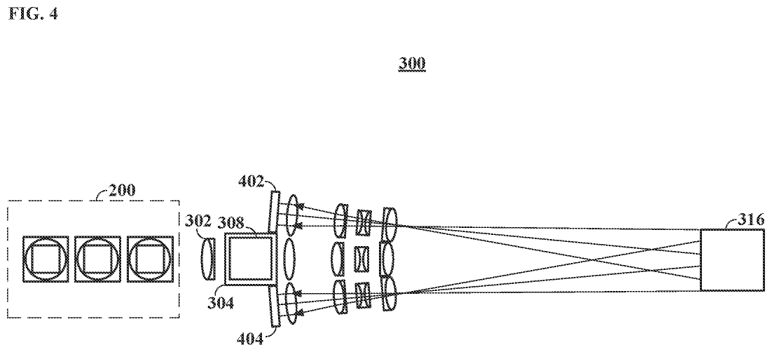

FIG. 4 depicts a top view of an embodiment of a multispectral scanner 300 according to this disclosure. The projection components may be as shown in FIG. 3, which are not fully labeled here again for clarity. In this embodiment, two imaging sensors 402 and 404 may be positioned on either side of the central illumination and projection path, though only one imaging sensor is required.

In one embodiment, imaging sensors 402 and 404 are each positioned at an offset angle of 4.5 degrees to either side of the illumination and projection path, as shown. This arrangement provides a stereo view of physical object 102 being observed, which can reduce ambiguity in observed data and also remedy any shadowing issues resulting from an uneven surface of physical object 102. Both advantages are helpful in producing a three-dimensional model of physical object 102 from the captured images.

In another embodiment, not shown, light may also be projected laterally from the sides of the scanner 300 toward the physical object 102. Images may also be captured from the sides of the physical object 102. Such side-lighting may help further reduce shadowing and may enhance observations of features best revealed via the longer-wavelength translucence of the physical object 102, such as cracks and caries. See, for example, commonly-assigned U.S. Pat. No. D918209S, which is hereby incorporated by reference in its entirety.

Imaging sensors 402 and 404 of this disclosure may comprise charge-coupled devices (CCDs) or color CMOS imaging sensors as may be known in the art. The resolution and image acquisition speed required for successful operation may be met by many such sensors available in today's marketplace. The imaging sensors used are preferably capable of generating images at least at conventional video frame rates, to simplify the conversion of images to video if necessary. Some exemplary CMOS imaging sensors used in this disclosure are capable of outputting 500 frames per second.

As noted, a single handheld scanning device 300 as disclosed may generate all of the required illumination and gather all of the required imagery for model generation in a substantially immediate time frame. Thus, there is no need to change scanning instruments while examining a patient, nor to cause related inconvenience to the patient or the users of the apparatus. Further, because all of the images may be captured practically simultaneously, the relative proximity and geometry between the scanning tip and physical object 102 remains effectively static during imaging.

The captured images and the computed three-dimensional model may be presented to a patient and/or a scanning tip user practically immediately after a scan, and may be stored for future use. The simultaneous presentation of images from multiple different illumination spectra may increase the likelihood of a correct diagnosis of a medical issue by a dentist or other medical practitioner. In another use scenario, the images may also comprise images of bones other than teeth, which may become directly observable with visible, infrared, and ultraviolet light during surgery, for example. This capability can help a surgeon ensure the soundness of bone structures that will receive various surgically implanted devices.

Note also that the use of non-x-ray illumination may reduce the risk of harm to a patient and users of scanner 300, such as staff in a dental office or operating theater, caused by x-rays. Also, the use of lead or other radiation shielding conventionally employed with x-ray imaging technologies becomes unnecessary with the technologies of this disclosure. The cost of acquiring and disposing of radiation shielding is not insignificant, particularly given the toxicity of lead.

In one embodiment, x-ray based images may however be superimposed onto the captured images from scanner 300 to provide diagnostic data from even shorter and more penetrating wavelengths than ultraviolet light. Such x-ray based images may comprise prior dental x-ray images of visible teeth in one embodiment. In another embodiment, the x-ray based images may comprise images of other bones that are not normally directly visible, such as those in which artificial joints or other medical devices have been or will be surgically implanted. Such image superimposition may be performed by an artificial intelligence (AI) program.

An AI program may also be trained to identify diagnostic indications from captured images, and to assist practitioners in noting possible problems requiring treatment. For example, many images of dental caries observed with different spectra may be used to train such a program to reliably recognize caries. Likewise, an AI may be trained to recognize and alert a patient and/or medical practitioners to other problems detectable from the imagery, such as intra-oral cancers, skin cancers, infected tissues, etc. Image processing of x-rays and conventional photographs with AI tools is well known in the art, and tools for such processing may be readily adapted to this use. Thus, the availability of multiple images made with different wavelengths of electromagnetic radiation can help provide a more complete diagnostic or investigative rendering of a medical situation.

Images may be acquired and three-dimensional models may be constructed therefrom both before a treatment and after a treatment, in one embodiment. This feature enables an evaluation of the treatment, i.e., to determine if the treatment successfully resolved an identified problem. For example, if a dental carie is identified by the scanner before treatment, a post-treatment multispectral view of the treated site may help determine if the entire depth of the carie has actually been drilled out completely and then fully filled. Such evidence could become significant for insurance, forensic, and litigation purposes.

Changes in the images taken over time even without a treatment may provide useful diagnostic information. For example, image comparisons could indicate the buildup of stains on a tooth surface that could cause a previously installed dental device to no longer match other teeth cosmetically as well as in the past. Likewise, image changes from one patient visit to the next could indicate the progression of cracks in teeth or worrisome changes in the gums or other oral tissues. Changes in the image-derived three-dimensional models built at different patient visits may also allow the precise determination of the movement and/or reorientation of teeth over time, to evaluate the effectiveness of orthodontic treatments, for example.

The availability of the multispectral images and the three-dimensional model derived from them essentially ‘on the spot’ also allows the patient and medical practitioners to examine a problem area in detail during the patient's office visit. For example, by rotating such a model of a tooth along with correspondingly rotated and overlaid projected multispectral image data on a high-resolution display, a patient could see a carie hidden inside a crevice or a space between teeth that would be difficult for the patient to observe directly. Such detailed review of medical issues and possible treatment approaches could encourage a patient to agree to a treatment plan the patient might otherwise forego, thus improving patient health.

Further, the physical condition relationships between multiple teeth may be more precisely understood with such a model-plus-imagery combination as may be provided by the teachings of this disclosure. Much of modern dentistry involves the re-working of teeth that have previously been treated, e.g., filling a carie invariably causes some long-term weakening of the filled tooth, which could cause other problems many years later. If a given previously-treated tooth is to be used as an anchor for bridgework or other dental appliances, for example, thoroughly ascertaining the current soundness of that anchor tooth is of heightened importance.

This disclosure also has particular utility entirely outside the dental treatment field. Physical objects of many kinds may be multispectrally imaged and three-dimensionally modeled using the systems and methods of this disclosure. In addition to determining precise changes to size, color, and shape of physical object 102 from one imaging session to another, it is possible to compare its size, color, and shape from one imaging session to a set of predetermined values.

Manufactured items are typically made to particular specifications of size, color, and shape, often including precise surface features and markings, and translucency characteristics. Quality control via comparison of various manufactured attributes versus designed attributes is often a complicated, expensive, and time-consuming activity. Yet quality control is crucial to many valuable economic activities, including but not limited to the manufacture of products in the aerospace, automotive, electronic, medical, and pharmaceutical fields, for example. The teachings of this disclosure enable manufacturing defects to be more readily investigated and identified on a production line. Even entirely counterfeit products may be detected in the marketplace more readily when a scan by the disclosed apparatus indicates their properties are not what they should be.

In another embodiment, the multispectral illumination may also comprise wavelengths specifically chosen for wavelength synthesis in holographic scanning. These wavelengths may be provided via an external illuminator and supplied to the scanning tip via coupled optical fibers. Details of such an illuminator are provided in U.S. patent application Ser. No. 18/455,027 previously mentioned. The construction of three-dimensional models may rely on either the patterned light approach, the holographic approach, or both.

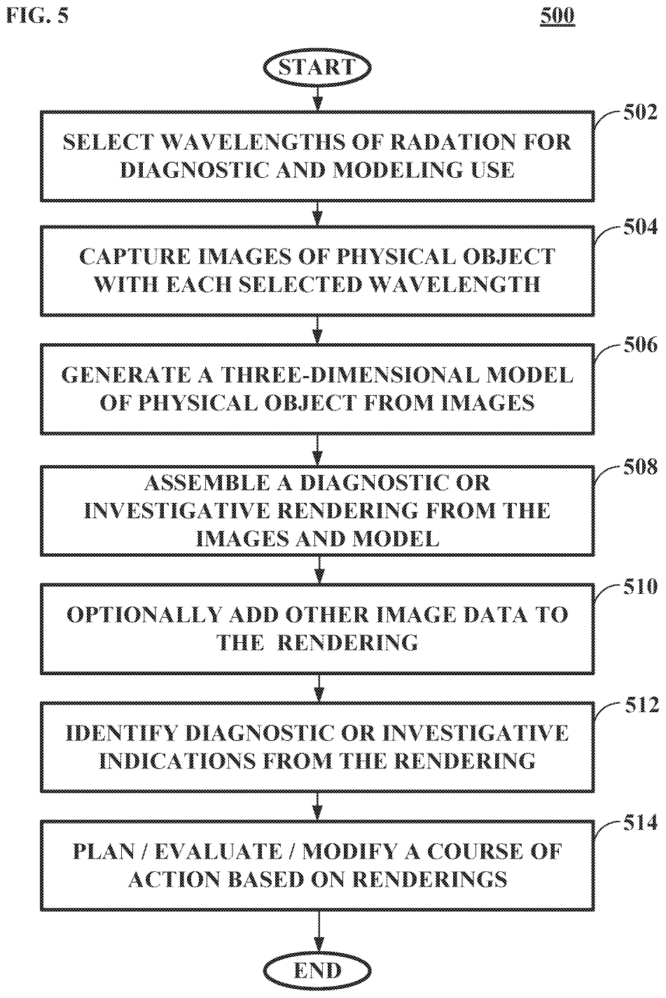

FIG. 5 depicts a flowchart 500 describing the operation of the scanning system 300 in which the techniques of this disclosure may be implemented. First, at 502, the scanner 300 selects wavelengths to be used to illuminate physical object 102. The wavelength selections are based on the diagnostic and modeling utility of the wavelengths. Next, at 504, the scanner 300 captures images of physical object 102 using each of the selected wavelengths.

Then, at 506, scanner 300 or a supporting computer system generates a three-dimensional model of physical object 102 from the captured images. The model may be generated using photogrammetry and triangulation, for example. At 508, scanner 300 or a supporting computer system assembles the images and model into a diagnostic or investigative rendering. The captured images may be projected onto the three-dimensional model, which may be presented on a high resolution display for detailed review by a viewer.

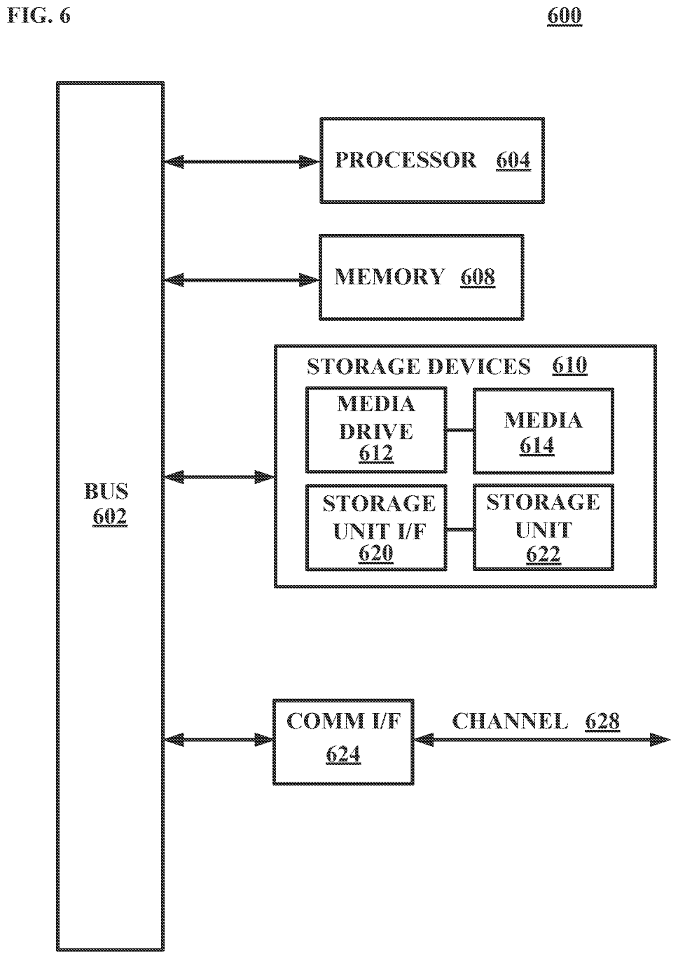

At 510, the scanner 300 or a supporting computer system may optionally add additional imagery to the diagnostic or investigative rendering. For example, previously existing dental x-rays may be added to images captured in an oral scan to add detail that might not otherwise be visible. At 512, the scanner 300 or a supporting computer system may identify diagnostic indications from the diagnostic rendering. For example, caries or cracks may be much more apparent in images made with wavelengths that penetrate and scatter in physical object 102, and so such images may be ideal for training examples for an AI engine. Likewise, familiar patterns indicating skin cancer, infection, or scar tissue may be recognized from the diagnostic rendering by either automated tools or a trained human medical practitioner, or both. Patterns indicating damaged, defective, or counterfeit products may also be recognized from the investigative rendering. Finally, at 514, the output of scanner 300 and the results of the modeling and image analyses, i.e., the diagnostic or investigative rendering and derived diagnostic or investigative indications, if any, may be used to plan, modify, or evaluate a course of action, such as a patient treatment or a product processing decision. In a dental scenario, the treatment may comprise a cleaning, a bleaching, repairing a carie, replacing a filling or other dental appliance, adjusting braces or retainers to move and reorient teeth, or replace a tooth with an implant, etc. In a quality control scenario, the product may be flagged for further examination or destruction.

Where components or components of the technology are implemented in whole or in part using software, in one embodiment, these software elements can be implemented to operate with a computing or processing component capable of carrying out the functionality described with respect thereto. One such example computing component is shown in FIG. 6. Various embodiments are described in terms of this example computing component 600. After reading this description, it will become apparent to a person skilled in the relevant art how to implement the technology using other computing components or architectures.

FIG. 6 shows a computing component that may carry out the functionality described herein, according to an embodiment. Computing component 600 may represent, for example, computing or processing capabilities found within desktop, laptop and notebook computers, hand-held computing devices (personal digital assistants (PDAs), smart phones, cell phones, palmtops, etc.), mainframes, supercomputers, workstations or servers, or any other type of special-purpose computing devices as may be desirable or appropriate for a given application or environment. Computing component 600 might also represent computing capabilities embedded within or otherwise available to a given device. For example, a computing component might be found in other electronic devices such as, for example, digital cameras, navigation systems, cellular telephones, portable computing devices, modems, routers, wireless application protocols (WAPs), terminals and other electronic devices that might include some form of processing capability.

Computing component 600 might include, for example, one or more processors, controllers, control components, or other processing devices, such as a processor 604. Processor 604 might be implemented using a special-purpose processing engine such as, for example, a microprocessor, controller, or other control logic. In the illustrated example, processor 604 is connected to a bus 602, although any communication medium can be used to facilitate interaction with other components of computing component 600 or to communicate externally.

Computing component 600 might also include one or more memory components, simply referred to herein as main memory 608. For example, random access memory (RAM) or other dynamic memory, might be used for storing information and instructions to be executed by processor 604. Main memory 608 might also be used for storing temporary variables or other intermediate information during execution of instructions to be executed by processor 604. Computing component 600 might likewise include a read only memory (ROM) or other static storage device coupled to bus 602 for storing static information and instructions for processor 604.

The computing component 600 might also include one or more various forms of information storage mechanism 610, which might include, for example, a media drive 612 and a storage unit interface 620. The media drive 612 might include a drive or other mechanism to support fixed or removable storage media 614. For example, a hard disk drive, a floppy disk drive, a magnetic tape drive, an optical disk drive, a compact disc (CD) or digital versatile disc (DVD) drive (read-only or read/write), or other removable or fixed media drive might be provided. Accordingly, storage media 614 might include, for example, a hard disk, a floppy disk, magnetic tape, cartridge, optical disk, a CD or DVD, or other fixed or removable medium that is read by, written to or accessed by media drive 612. As these examples illustrate, the storage media 614 can include a computer usable storage medium having stored therein computer software or data.

In alternative embodiments, information storage mechanism 610 might include other similar instrumentalities for allowing computer programs or other instructions or data to be loaded into computing component 600. Such instrumentalities might include, for example, a fixed or removable storage unit 622 and an interface 620. Examples of such storage units 622 and interfaces 620 can include a program cartridge and cartridge interface, a removable memory (for example, a flash memory or other removable memory component) and memory slot, a personal computer memory card international association (PCMCIA) slot and card, and other fixed or removable storage units 622 and interfaces 620 that allow software and data to be transferred from the storage unit 622 to computing component 600.

Computing component 600 might also include a communications interface 624. Communications interface 624 might be used to allow software and data to be transferred between computing component 600 and external devices. Examples of communications interface 624 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 624 might typically be carried on signals, which can be electronic, electromagnetic (which includes optical) or other signals capable of being exchanged by a given communications interface 624. These signals might be provided to communications interface 624 via a channel 628. This channel 628 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 608, storage unit 620, media 614, and channel 628. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 600 to perform features or functions of the disclosed technology as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent component names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any tune in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The Abstract of the Disclosure is provided to comply with 37 C.F.R. § 1.72 (b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

The foregoing description has outlined some of the more pertinent features of the subject matter. These features should be construed to be merely illustrative.

Having described the various embodiments, what is claimed is as follows.

The invention claimed is:

1. A method for producing a diagnostic rendering of a patient's anatomy, comprising:
selecting a plurality of wavelengths of electromagnetic radiation for at least one of diagnostic and modeling characteristics;
capturing images of the patient's anatomy when illuminated using the selected wavelengths;
generating a three-dimensional model of the patient's anatomy using at least some of the captured images; and
assembling the diagnostic rendering from at least some of the captured images and the model.

2. The method of claim 1, wherein the patient's anatomy comprises at least one of a tooth, intra-oral tissue, and skin.

3. The method of claim 1, further comprising basing the selecting of at least one wavelength on a translucency of the patient's anatomy resulting from illumination with the selected wavelength.

4. The method of claim 1, further comprising basing the selecting of at least one wavelength on a fluorescence of the patient's anatomy resulting from illumination with the selected wavelength.

5. The method of claim 1, wherein the capturing and the generating are substantially simultaneous.

6. The method of claim 1, wherein the generating is based on a photogrammetric analysis of patterned illuminations of the patient's anatomy.

7. The method of claim 1, wherein the generating is based on a hologram of the patient's anatomy.

8. The method of claim 1, further comprising mapping at least some of the captured images onto the three-dimensional model to describe a treatment plan.

9. The method of claim 1, wherein the assembling further comprises mapping x-ray images of the patient's anatomy onto the model.

10. The method of claim 1, further comprising identifying diagnostic indications from the diagnostic rendering.

11. The method of claim 10, further comprising:
evaluating a treatment by comparing the diagnostic renderings captured after the treatment to the diagnostic renderings captured before the treatment.

12. The method of claim 10, further comprising:
storing diagnostic renderings from different times;
comparing at least a plurality of the stored diagnostic renderings; and
identifying the diagnostic indications from the compared diagnostic renderings.

13. The method of claim 10, further comprising performing at least one of the assembling and the identifying using an artificial intelligence engine.

14. An apparatus for producing a diagnostic rendering of a patient's anatomy, comprising:
an illuminator configured to generate electromagnetic radiation of a plurality of wavelengths selected for at least one of diagnostic and modeling characteristics;
at least one imaging sensor configured to capture images of the patient's anatomy when illuminated with the selected wavelengths; and
a modeling engine configured to generate a three-dimensional model of the patient's anatomy using at least some of the captured images and to assemble the diagnostic rendering from at least some of the captured images and the model.

15. The apparatus of claim 14, wherein the patient's anatomy comprises at least one of a tooth, intra-oral tissue, and skin.

16. The apparatus of claim 14, wherein the illuminator emits at least one wavelength based on a translucency of the patient's anatomy resulting from illumination with the selected wavelength.

17. The apparatus of claim 14, wherein the illuminator emits at least one wavelength based on a fluorescence of the patient's anatomy resulting from illumination with the selected wavelength.

18. The apparatus of claim 14, wherein the capturing and generating are substantially simultaneous.

19. The apparatus of claim 14, wherein the modeling engine generates the model based on a photogrammetric analysis of patterned illuminations of the patient's anatomy.

20. The apparatus of claim 14, wherein the modeling engine generates the model based on a hologram of the patient's anatomy.

21. The apparatus of claim 14, wherein the modeling engine maps x-ray images of the patient's anatomy onto the model.

22. The apparatus of claim 14 wherein the illuminator and the imaging sensor are housed in a single handheld scanning tip.

23. The apparatus of claim 14, further comprising an analysis engine configured to identify diagnostic indications from the diagnostic rendering.

24. The apparatus of claim 23, wherein the analysis engine evaluates a treatment by comparing the diagnostic renderings captured after the treatment to the diagnostic renderings captured before the treatment.

25. The apparatus of claim 23, wherein the analysis engine compares at least a plurality of stored diagnostic renderings from different times and identifies the diagnostic indications from the compared diagnostic renderings.

26. The apparatus of claim 23, wherein an artificial intelligence engine performs at least one of the assembling and the identifying.

27. The apparatus of claim 14 wherein the illuminator generates radiation with at least one of laser diodes and light-emitting diodes.

28. The apparatus of claim 27 wherein at least one of a light-emitting diode and a set of laser diodes of different spectra emits substantially white light for capturing a full-color image.

29. A system for producing a diagnostic rendering of a patient's anatomy, comprising:
means for selecting a plurality of wavelengths of electromagnetic radiation for at least one of diagnostic and modeling characteristics;
means for capturing images of the patient's anatomy when illuminated using the selected wavelengths;
means for generating a three-dimensional model of the patient's anatomy using at least some of the captured images; and
means for assembling the diagnostic rendering from at least some of the captured images and the model.

30. A computer program product comprising a non-transitory computer-readable medium with computer-executable instructions tangibly embodied thereon that, when executed by a processor, produce a diagnostic rendering of a patient's anatomy by:
selecting a plurality of wavelengths of electromagnetic radiation for at least one of diagnostic and modeling characteristics;
capturing images of the patient's anatomy when illuminated using the selected wavelengths;
generating a three-dimensional model of the patient's anatomy using at least some of the captured images; and
assembling the diagnostic rendering from at least some of the captured images and the model.
